(19) United States
(12) Patent Application Publication
(10) Pub. No.: US 2008/0171746 A1
(43) Pub. Date: Jul. 17, 2008

(54) Method for Preventing Cardiovascular Diseases

(76) Inventors: Thomas Klein, Konstanz (DE); Manfrid Eltze, Konstanz (DE); Armin Hatzelmann, Konstanz (DE)

Correspondence Address: NATH & ASSOCIATES PLLC, 112 South West Street, Alexandria, VA, 22314, US

(21) Appl. No.: 11/885,836

(22) PCT Filed: Mar. 14, 2006
(86) PCT No.: PCT/EP06/60686

§ 371 Date: Sep. 28, 2007

Related U.S. Application Data
(60) Provisional application No. 60/659,791, filed on Mar. 14, 2005.

(30) Foreign Application Priority Data
Mar. 14, 2005 (EP) 05101977.6

(51) Int. Cl. A61K 31/53; A61K 31/415; A61K 31/44; A61K 31/19; A61K 31/42; A61P 9/00; A61K 31/34; A61K 31/496; A61K 31/4985

(52) U.S. Cl. 514/243; 514/406; 514/334; 514/557; 514/378; 514/473; 514/252.16; 514/250

(57) ABSTRACT

The invention relates to a method for selecting COX-2 selective NSAIDs which have a salutary therapeutic profile. The invention further relates to the use of these selected COX-2 selective NSAIDs in the treatment of certain diseases. The invention yet further relates to combinations comprising COX-2 selective NSAIDs and the use of these combinations.

### FIELD OF APPLICATION OF THE INVENTION

The invention relates to a method for selecting COX-2 selective NSAIDs which have a salutary therapeutic profile. The invention further relates to the use of these selected COX-2 selective NSAIDs in the treatment of certain diseases. The invention yet further relates to combinations comprising COX-2 selective NSAIDs and the use of these combinations.

### TECHNICAL BACKGROUND

Cyclooxygenases catalyse a rate-limiting step in the prostaglandin synthesis casacade. The prostaglandins produced are major mediators in inflammatory and physiological processes. The discovery of novel selective COX-2 inhibitors led to a better gastro-intestinal safety of these class compared to unselective NSAIDs. However, recent data from clinical studies demonstrated despite the better GI profile an increased cardio-vascular risk of Coxibs. An explanation of this observation could be the un-opposed inhibition of the major antiaggregational and vasodilatory prostanoid Prostacyclin, whereas the essentially prothrombotic prostanoid Thromboxane was not affected.

Another potent vasodilator and antithrombotic agent is nitric oxide (NO). The physiological effect of NO are mediated via soluble guanylate cyclase, which in turn produces cyclic GMP. The PDE5 is the most predominant enzyme responsible for cGMP degradation. Thus, PDE5 inhibition could strengthen the effect of NO and thereby compensate a reduced production of Prostacyclin.

### DESCRIPTION OF THE INVENTION

The use of nonsteroidal anti-inflammatory drugs (NSAIDs), particularly of certain selective cyclooxygenase 2 (COX-2) inhibitors (such as e.g. certain coxibs), can be associated with the risk of renal or cardiovascular diseases, e.g. cardiovascular adverse events, such as myocardial infarction or cerebrovascular accidents, which limit their widespread clinical use.

Investigations into the role of several NSAIDs in inhibiting the phosphodiesterase 5 (PDE5 ) in vitro show that from the class of NSAIDs with preferential COX-2 selectivity some compounds (such as, for example, nimesulide [INN], flosulide [INN], CGP28237 [Research Code], L-745337 [Research Code], or, and in particular, the highly selective drug celecoxib [INN]) possess PDE5 activity in the μM range, whereas other compounds from this class (such as, for example, lumiracoxib [INN], rofecoxib [INN], valdecoxib [INN], parecoxib [INN] and etorecoxib [INN]) and conventional NSAIDs (for example, acetylsalicylic acid, diclofenac [INN]) show no effects.

In isolated guinea pig Langendorff hearts, the PDE5 inhibitory component of celecoxib is reflected by a selective increase in coronary heart flow, but with no effect on left ventricular pressure and heart rate, thereby excluding inhibition of PDE3. In this model, lumiracoxib, valdecoxib, rofecoxib, parecoxib, etorecoxib and diclofenac compared with celecoxib substantially lack the ability to increase coronary heart flow and therefore PDE5 inhibition. These results illustrate that some NSAIDs with preferential COX-2 selectivity (i.e. COX-2 selective NSAIDs), particularly some coxibs, can increase coronary heart flow correlated with their PDE5-inhibitory activity. This together with an antithrombotic action mediated via PDE5 inhibition may result in the reduction of the risk of cardio-renal diseases.

As useful consequence of these findings it is now possible to identify and provide agents having a superior therapeutic index vis-a-vis COX-2 inhibitory and anti-inflammatory activities versus cardiovascular and/or renal side effects.

As one aspect (aspect a) of the present invention, one can say that the intrinsic PDE5-inhibitory component of COX-2 selective NSAIDs most likely contribute to the cardiovascular and/or renal safety of these drugs.

The invention thus relates to the use of a compound from the class of COX-2 selective NSAIDs, such as e.g. from the coxib class, having, as further property within the same molecule, an intrinsic PDE5-inhibitory component in the manufacture of a pharmaceutical composition for the prevention of cardiovascular and/or renal side effects customary associated with the use of COX-2 selective NSAIDs.

The invention also relates to the use of a compound from the class of COX-2 selective NSAIDs, such as e.g. from the coxib class, having an intrinsic PDE5-inhibitory component in the manufacture of a pharmaceutical composition for the prevention of cardiovascular diseases, such as e.g. cardiovascular diseases customary associated with the use of COX-2 selective NSAIDs.

The invention also relates to the use of a compound from the class of COX-2 selective NSAIDs, such as e.g. from the coxib class, having an intrinsic PDE5-inhibitory component in the manufacture of a pharmaceutical composition for use in the therapy or prophylaxis of diseases responsive to COX-2 inhibition.

The invention also relates to the use of a compound from the class of COX-2 selective NSAIDs, such as e.g. from the coxib class, having an intrinsic PDE5-inhibitory component in the manufacture of a pharmaceutical composition for use in cardio-protective therapy or prophylaxis of diseases responsive to COX-2 inhibition.

The invention also relates to the use of a compound from the class of COX-2 selective NSAIDs, such as e.g. from the coxib class, having an intrinsic PDE5-inhibitory component in the manufacture of a pharmaceutical composition for use in the therapy or prophylaxis of diseases responsive to COX-2 inhibition in a patient group at risk of cardiovascular diseases.

The invention also relates to the use of a compound from the class of COX-2 selective NSAIDs, such as e.g. from the coxib class, having an intrinsic PDE5-inhibitory component in the manufacture of a pharmaceutical composition for use in the long-term therapy or prophylaxis of diseases responsive to COX-2 inhibition.

The invention also relates to the use of a compound from the class of COX-2 selective NSAIDs, such as e.g. from the coxib class, having an intrinsic PDE5-inhibitory component in the manufacture of a pharmaceutical composition for use in increased- or high-dose therapy or prophylaxis of diseases responsive to COX-2 inhibition.

The invention also relates to a method for inhibiting selectively (preferentially) COX-2 while reducing the risk of cardiovascular diseases comprising administering to a mammal in need thereof a effective amount of a compound from the class of COX-2 selective NSAIDs, such as e.g. from the coxib class, which has an intrinsic PDE5-inhibitory component.

The invention also relates to a method for treating, preventing or ameliorating of a disease responsive to COX-2 inhibition comprising administering to a mammal, including a human, in need thereof a therapeutically effective amount of a compound from the class of COX-2 selective NSAIDs, such as e.g. from the coxib class, which has an intrinsic PDE5-inhibitory component.

The invention also relates to a method for treating, preventing or ameliorating a disease responsive to COX-2 inhibition while reducing the risk of cardiovascular and/or renal side-effects associated with therapeutic use of COX-2 selective NSAIDs in a mammal, including a human, in need thereof comprising administering to said mammal a therapeutically effective and tolerable amount of a compound from the class of COX-2 selective NSAIDs, such as e.g. from the coxib class, having an intrinsic PDE5-inhibitory component.

The invention also relates to a method for treating, preventing or ameliorating a disease responsive to COX-2 inhibition while reducing the risk of cardiovascular diseases in a mammal, including a human, in need thereof comprising administering to said mammal a therapeutically effective and tolerable amount of a compound from the class of COX-2 selective NSAIDs, such as e.g. from the coxib class, having an intrinsic PDE5-inhibitory component.

The invention also relates to a method for treating, preventing or ameliorating a disease responsive to COX-2 inhibition and reducing the risk of cardiovascular diseases in a mammal, including a human, in need of such treatment, prevention or amelioration and at risk of cardiovascular diseases comprising administering to said mammal a therapeutically effective and tolerable amount of a compound from the class of COX-2 selective NSAIDs, such as e.g. from the coxib class, having an intrinsic PDE5-inhibitory component.

The invention also relates to a method for preventing cardiovascular diseases in a mammal, including human, in need of such prevention comprising administering a therapeutically effective and tolerable amount of a compound from the class of COX-2 selective NSAIDs, such as e.g. from the coxib class, having an intrinsic PDE5-inhibitory component.

The invention also relates to a method for treating, preventing or ameliorating a disease responsive to COX-2 inhibition in a subject by administering to a patient in need thereof a COX-2 selective NSAID, which method comprises administering to a mammal, including human, in need thereof a therapeutically effective and tolerable amount of a COX-2 selective NSAID, such as e.g. from the coxib class, having an intrinsic PDE5-inhibitory component.

The invention also relates to a method for long-term treating, preventing or ameliorating a disease responsive to COX-2 inhibition in a subject by administering to a patient in need thereof a COX-2 selective NSAID, which method comprises administering to a mammal, including human, in need thereof a therapeutically effective and tolerable amount of a COX-2 selective NSAID, such as e.g. from the coxib class, having an intrinsic PDE5-inhibitory component.

The invention also relates to a method for treating, preventing or ameliorating a disease responsive to COX-2 inhibition in a subject by administering to a patient in need thereof a COX-2 selective NSAID, which method comprises administering to a mammal, including human, in need thereof an increased or high amount of a COX-2 selective NSAID, such as e.g. from the coxib class, having an intrinsic PDE5-inhibitory component.

By the expression COX-2 selective NSAID is meant herein a selective COX-2 inhibitor, which preferentially inhibits the cyclooxygenase-2 (COX-2) when compared to cyclooxygenase-1 (COX-1). In one embodiment, the compound has a cyclooxygenase-2 IC₅₀ of less than about 2 μM and a cyclooxygenase-1 IC₅₀ of greater than about 5 μM, in the human whole blood COX-2 assay (as described in Brideau et al., Inflamm Res., 45: 68-74 (1996)) and also has a selectivity ratio of cyclooxygenase-2 inhibition over cyclooxygenase-1 inhibition of at least 10, and preferably of at least 40. In another embodiment, the compound has a cyclooxygenase-1 IC₅₀ of greater than about 1 μM, and preferably of greater than 20 μM, and/or a cyclooxygenase-2 IC₅₀ of less than about 1 μM, preferably less than about 0.5 μM, and more preferably less than about 0.2 μM.

It is to be understood, that in the meaning of this invention, a COX-2 selective NSAID, such as e.g. from the coxib class, having an intrinsic PDE5-inhibitory component refers to a molecule which should have in general, additional to its COX-2 inhibiting activity, a capability, within the same molecule, to inhibit the PDE5 activity in the range of the its COX-2 inhibiting potency. Advantageously, the IC50 range of inhibiting PDE5 activity may be hereby in the nmolar range, but also inhibition of the PDE5 activity in the μmolar range, particularly less than 50 μM (depending on the respective test system used), will be sufficient to demonstrate beneficials effects.

Suitable systems for detecting said intrinsic PDE5-inhibitory activity are recombinant enzymes or cellular systems containing high amounts of PDE5 such as thrombocytes or any other in vitro and in vivo models which reflects inhibition of PDE5 activity via physiological parameters e.g. coronary flow in the langendorf heart models etc.

It is to be noted, that in the meaning of this invention, a COX-2 selective NSAID, such as e.g. from the coxib class, having an intrinsic PDE5-inhibitory component refers to one compound having two different properties namely that of a COX-2 selective NSAID and that of a PDE5 inhibitor.

As cardiovascular diseases according to this invention can be mentioned, for example, cardiovascular or thromboembolic adverse events, such as myocardial infarction or cerebrovascular accidents, e.g. heart attack or stroke, or cardiovascular diseases which can be customary associated with the use of COX-2 selective NSAIDs lacking an intrinsic PDE5-inhibitory component, such as e.g. those diseases mentioned afore, which limit their widespread clinical use.

As diseases responsive to COX-2 inhibition according to this invention can be mentioned diseases which can be treated, prevented or ameliorated by a COX-2 inhibitor, such as e.g. without being restricted thereto, acute or chronic inflammatory diseases (in particular all kind of arthritis including rheumatoid arthritis or degenerative joint diseases including osteoarthritis) or inflammation associated disorders, and/or particularly symptoms caused by arthritis, such as inflammation, swelling, stiffness and joint pain, or other kinds of acute or chronic pain or painful conditions, such as e.g. gout attacks, bursitis, tendonitis, toothache, migraine, lower back and neck pain, myositis, menstrual cramps, sprains, strains or other injuries, or symptoms associated with influenza or other viral infections or common cold; as well as neuropathic pains, (inflammatory) liver diseases, stroke, epilepsy, dysmenorrhoea, ophthalmic diseases, cognitive disorders such as dementia, degenerative dementia (such e.g. Alzheimer's disease), or cellular and neoplastic transformation and metastatic tumour growth, such e.g. certain cancerous diseases, for example colonic cancer and prostate cancer, or cancer associated with overexpression of HER-2/neu (e.g. breast cancer), or adenomatous colorectal polyps (and to reduce herewith the risk of developing colon cancer), or other conditions mediated by COX-2 (such as, e.g. conditions mediated by COX-2 overexpression during carcinogenesis).

In this context, particular suitable diseases or conditions responsive to COX-2 inhibition which may be treated, prevented or ameliorated by the use of COX-2 selective NSAID (such as e.g. from the coxib class) having an intrinsic PDE5-inhibitory component in the meaning of this invention are those from chronic nature, such as, for example, chronic inflammatory diseases (particularly all kind of arthritis including rheumatoid arthritis or degenerative joint diseases including osteoarthritis), or Alzheimer's disease, or certain cancerous or pre-cancerous diseases, such as e.g. colorectal adenoms or polyps (e.g. prevention of spontaneous adenomatopus polyps or adenoma prevention).

Further on in this context, particular suitable diseases or conditions responsive to COX-2 inhibition which may be treated, prevented or ameliorated by the use of COX-2 selective NSAID (such as e.g. from the coxib class) having an intrinsic PDE5-inhibitory component in the meaning of this invention are those, which are treated, prevented or ameliorated with an increased or high dose thereof, and/or those, which are treated, prevented or ameliorated therewith over long term, and/or those, which are treated, prevented or ameliorated therewith in a patient group at risk of cardiovascular diseases, such as e.g. those diseases or conditions which are from chronic nature, e.g. those as mentioned afore.

Further, as another aspect (aspect b) of the present invention, an useful significance of the finding disclosed in this invention is that it is feasible to reduce the risk of cardio-renal diseases associated with the use of COX-2 selective NSAIDs by designing or choosing COX-2 selective NSAIDs which show an intrinsic PDE5-inhibitory component.

The invention thus relates to a method for identifying a COX-2 selective NSAID useful for treating diseases responsive to COX-2 inhibition, such as e.g. inflammatory diseases, while reducing the risk of cardiovascular diseases, such as e.g. cardiovascular adverse events, which method comprises determining for said COX-2 selective NSAID the existence of an intrinsic PDE5-inhibitory component.

The invention further relates to the use of an intrinsic PDE5-inhibitory component as an integral characteristic of compounds which inhibit selectively COX-2, such as e.g. coxibs.

The invention further relates to a method of treating a disease responsive to COX-2 inhibition in a patient comprising administering to said patient a therapeutically affective amount of a COX-2 selective NSAID, such as e.g. a coxib, selected by determining PDE5 inhibitory activity of said COX-2 selective NSAID, wherein the COX-2 selective NSAID that is selected for administration to said patient inhibits PDE5 activity in the range of its COX-2 inhibiting potency.

The invention further relates to a method of treating a disease responsive to COX-2 inhibition in a patient comprising administering to said patient a therapeutically affective amount of a COX-2 selective NSAID, such as e.g. a coxib, selected by determining PDE5 inhibitory activity of said COX-2 selective NSAID, wherein the COX-2 selective NSAID that is selected for administration to said patient inhibits PDE5 activity less than about 50 μM, in another embodiment less than about 20 μM.

The invention further relates to a method of treating a disease responsive to COX-2 inhibition in a patient comprising administering to said patient a therapeutically affective amount of a compound selected by:

determining COX inhibitory activity of said compound, and
determining PDE5 inhibitory activity of said compound,
wherein the compound that is selected for administration to said patient has a COX-1 IC₅₀ of greater than about 1 μM, preferably greater than about 20 μM, a COX-2 IC₅₀ of less than about 1 μM, preferably less than about 0.5 μM, and inhibits PDE5 activity less than about 20 μM or, in another embodiment, less than about 10 μM, or, in yet another embodiment, less than about 1 μM.

The present invention covers those COX-2 selective NSAIDs, which have an intrinsic PDE5-inhibitory component. This includes any and all compounds which are COX-2 selective NSAIDs, e.g. as per the test set out herein, and which demonstrate in the herein-given or similar assays, an intrinsic PDE5-inhibitory component as defined above; of particular interest are those compounds which are not in the public domain and/or not tested as or known to be COX-2 selective NSAIDs having an intrinsic PDE5-inhibitory component prior to the filing date of this application.

The present invention further relates to COX-2 selective NSAIDs having an intrinsic PDE5-inhibitory component, as well as the pharmaceutically acceptable derivatives (such as e.g. salts, esters, hydrates, polymorphs or stereoisomers) thereof.

The present invention further relates to a pharmaceutical composition comprising a COX-2 selective NSAID having an intrinsic PDE5-inhibitory component and a pharmaceutically acceptable carrier, vehicle or adjuvant.

Exemplary COX-2 selective NSAIDs having an intrinsic PDE5-inhibitory component which may be mentioned include the following compounds, without limiting the invention thereto:

nimesulide, CGP28238, L-745337 and celecoxib.

Exemplary COX-2 selective NSAIDs having an intrinsic PDE5-inhibitory component to be emphasized include the following compound, without limiting the invention thereto:

celecoxib.

The term “pharmaceutically acceptable salts” embraces salts commonly used to form alkali metal salts and to form addition salts of free acids or free bases. The nature of the salt may vary, provided that it is pharmaceutically acceptable. Suitable pharmaceutically acceptable acid addition salts of compounds for use in the present methods may be prepared from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, araliphatic, heterocyclic, carboxylic and sulfonic classes of organic acids, examples of which are formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, 4-hydroxybenzoic, phenylacetic, mandelic, embonic (pamoic), methanesulfonic, ethanesulfonic, benzenesulfonic, pantothenic, 2-hydroxyethanesulfonic, toluenesulfonic, sulfanilic, cyclohexylaminosulfonic, stearic, algenic, (3-hydroxybutyric, salicylic, galactaric and galacturonic acid. Suitable pharmaceutically acceptable base addition salts of compounds of use in the present methods include metallic salts made from aluminum, calcium, lithium, magnesium, potassium, sodium and zinc or organic salts made from N,N′-dibenzylethylenediamine, chloroprocaine, choline, diethanolamine, ethylenediamine, meglumine (N-methylglucamine) and procaine. All of these salts may be prepared by conventional means from the corresponding compound by reacting, for example, the appropriate acid or base with the compound according to this invention.

The compounds of this invention, i. e. the COX-2 selective NSAIDs having an intrinsic PDE5-inhibitory component, as well as the pharmaceutically derivatives thereof, can be formulated into pharmaceutical compositions and administered by any means that will deliver a therapeutically effective dose. Such compositions can be administered orally, parenterally, by inhalation spray, rectally, intradermally, transdermally, or topically in dosage unit formulations containing conventional nontoxic pharmaceutically acceptable carriers, adjuvants, and vehicles as desired. Topical administration may also involve the use of transdermal administration such as transdermal patches or iontophoresis devices. The term parenteral as used herein includes subcutaneous, intravenous, intramuscular, or intrasternal injection, or infusion techniques. A controlled release preparation can also be utilized. Formulation of drugs is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

Preferably, the pharmaceutical compositions comprising the compounds of this invention are adapted for oral or parenteral (especially oral) administration. Intravenous and oral, first and foremost oral, administration is considered to be of particular importance.

Injectable preparations, for example, sterile injectable aqueous or oleaginous suspensions, can be formulated according to the known art using suitable dispersing or wetting agents and suspending agents. The sterile injectable preparation may also be a sterile injectable solution or suspension in a nontoxic parenterally acceptable diluent or solvent. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed, including synthetic mono-or diglycerides. In addition, fatty acids such as oleic acid are useful in the preparation of injectables. Dimethyl acetamide, surfactants including ionic and non-ionic detergents, and polyethylene glycols can be used. Mixtures of solvents and wetting agents such as those discussed above are also useful.

Suppositories for rectal administration of the compounds discussed herein can be prepared by mixing the active agent with a suitable non-irritating excipient such as cocoa butter, synthetic mono-, di-, or triglycerides, fatty acids, or polyethylene glycols which are solid at ordinary temperatures but liquid at the rectal temperature, and which will therefore melt in the rectum and release the drug.

Solid dosage forms for oral administration may include capsules, tablets, pills, powders, and granules. In such solid dosage forms, the compounds are ordinarily combined with one or more adjuvants appropriate to the indicated route of administration. If administered per os, the compounds can be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets can contain a controlled-release formulation as can be provided in a dispersion of active compound in hydroxypropylmethyl cellulose. In the case of capsules, tablets, and pills, the dosage forms can also comprise buffering agents such as sodium citrate, or magnesium or calcium carbonate or bicarbonate. Tablets and pills can additionally be prepared with enteric coatings.

For therapeutic purposes, formulations for parenteral administration can be in the form of aqueous or non-aqueous isotonic sterile injection solutions or suspensions.

These solutions and suspensions can be prepared from sterile powders or granules having one or more of the carriers or diluents mentioned for use in the formulations for oral administration. The compounds can be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

Liquid dosage forms for oral administration can include pharmaceutically acceptable emulsions, solutions, suspensions, syrups, and elixirs containing inert diluents commonly used in the art, such as water. Such compositions can also comprise adjuvants, such as wetting agents, emulsifying and suspending agents, and sweetening, flavoring, and perfuming agents.

Typical dermal and transdermal formulations comprise a conventional aqueous or non-aqueous vehicle, for example, a cream, ointment, lotion or paste or are in the form of a medicated plaster, patch or membrane.

The dosage of the active compound can depend on a variety of factors, such as mode of administration, homeothermic species, body weight, age and/or individual condition.

In general, the pharmaceutical compositions may contain a compound according to this invention in the range of about 0.1 to 2000 mg, preferably in the range of about 0.5 to 500 mg and most preferably between about 1 and 200 mg. A daily dose of about 0.01 to 100 mg/kg body weight, preferably between about 0.1 and about 50 mg/kg body weight and most preferably from about 1 to 20 mg/kg body weight, may be appropriate. The daily dose can be administered in one to four doses per day.

When the COX-2 selective NSAID having an intrinsic PDE5-inhibitory component is CELECOXIB, it is preferred that the amount used is within a range of from about 1 to about 20 mg/kg per day, even more preferably from about 1.4 to about 8.6 mg/kg per day, and yet more preferably from about 2 to about 3 mg/kg per day. For example, doses of celecoxib for the treatment of osteoarthritis may be 100 to 200 mg per day, for the treatment of arthritis may be 200 to 400 mg per day, for the prevention of spontaneous adenomatopus polyps may be 400 mg a day, for adenoma prevention may be 400 to 800 mg a day, and for prevention of Alzheimer's disease may be 400 mg per day.

Those skilled in the art will appreciate that dosages may also be determined with guidance from Goodman & Goldman's The Pharmacological Basis of Therapeutics, Ninth Edition (1996), Appendix II, pp. 1707-1711 and from Goodman & Goldman's The Pharmacological Basis of Therapeutics, Tenth Edition (2001), Appendix II, pp. 475-493.

The present compounds are useful for the treatment or prophylaxis of diseases responsive to COX-2 inhibition, e.g. inflammatory diseases, such as e.g. those mentioned above.

In a special embodiment, the present compounds are particularly useful for the therapy or prophylaxis of the diseases mentioned herein while minimizing side effects commonly associated with standard therapy, such as e.g. cardio-renal side effects.

In a further special embodiment, the present compounds are particularly suited for use in a patient group with a non-acceptable risk (e.g. with a severe or high risk) for cardiovascular diseases, such as e.g. a patient group with an increased risk of myocardial infarction and stroke, such as e.g. patients with fluid retention, hypertension, dyslipidemia, preexisting cardiovascular diseases and/or heart failure.

As it is known for the skilled person, the risk for cardiovascular diseases can vary for each single patient depending, for example, from the individual susceptibility, therapeutic state of the patient, the history of prior cardiovascular diseases, serious systemic co-morbidities, co-medication, duration of therapy, and the like.

In a further special embodiment, the present compounds are particularly useful in long-term therapy.

In a further special embodiment, the present compounds are particularly useful in increased- or high-dose therapy.

In a further special embodiment, the present compounds are particularly useful in increased-dose therapy over long term.

No unacceptable toxicological effects, such as e.g. cardio-toxicological effects, are expected when present compounds are administered in accordance with the present invention.

### Combinations

Further on, as yet another aspect (aspect c) of the present invention, the risk of cardiovascular and/or renal diseases associated with the use of a COX-2 selective NSAID can be reduced if said COX-2 selective NSAID is applied in combination with a PDE5 inhibitor.

The invention thus relates to the combined use of a PDE5 inhibitor and a COX-2 selective NSAID, particularly a coxib, in the treatment of a disease responsive to COX-2 inhibition while minimizing the risk of cardiovascular diseases.

“Combined use” or “in combination with” in the context of this invention means the simultaneous, sequential, separate or chronologically staggered administration of the COX-2 selective NSAID on the one hand and of the PDE5 inhibitor on the other hand (such as e.g. as combined unit dosage forms, as separate unit dosage forms, as adjacent discrete unit dosage forms, as fixed or non-fixed combinations, as kit-of-parts or as admixtures).

The present invention further relates to a combination comprising

a first active ingredient, which is at least one COX-2 selective NSAID, such as e.g. a coxib, and
a second active ingredient, which is at least one PDE5 inhibitor,
for separate, sequential, simultaneous or chronologically staggered use in therapy, such as e.g. in therapy of diseases responsive to COX-2 inhibition, particularly those diseases mentioned herein.

The present invention further relates to a combination comprising

a first active ingredient, which is at least one COX-2 selective NSAID, such as e.g. a coxib, and
a second active ingredient, which is at least one PDE5 inhibitor,
for separate, sequential, simultaneous or chronologically staggered use in therapy, such as e.g. in prevention of cardiovascular diseases, like those mentioned above.

By the expression “COX-2 selective NSAID” according to aspect c of this invention is meant a selective COX-2 inhibitor, which preferentially inhibits the cyclooxygenase-2 (COX-2) when compared to cyclooxygenase-1 (COX-1). In one embodiment, the compound has a cyclooxygenase-2 IC₅₀ of less than about 2 μM and a cyclooxygenase-1 IC₅₀ of greater than about 5 μM, in the human whole blood COX-2 assay (as described in Brideau et al., Inflamm Res., 45: 68-74 (1996)) and also has a selectivity ratio of cyclooxygenase-2 inhibition over cyclooxygenase-1 inhibition of at least 10, and preferably of at least 40. In another embodiment, the compound has a cyclooxygenase-1 IC₅₀ of greater than about 1 μM, and preferably of greater than 20 μM, and/or a cyclooxygenase-2 IC₅₀ of less than about 1 μM, preferably less than about 0.5 μM, and more preferably less than about 0.2 μM.

As COX-2 selective NSAIDs within the meaning of aspect c of present invention may be mentioned, for example, without being limited to, ABT-963, BMS-347070, CS-402, CS-706, E-6087, FK-3311, GR-253035, GW-406381, L-745337, L-752860, LAS-33815, LAS-34475, PH-686464, SC-58236, SVT-2016, and the coxibs, such as e.g. celecoxib, cimicoxib, etoricoxib, firocoxib, lumiracoxib, parecoxib, rofecoxib, tilnmacoxib and valdecoxib, in particular celecoxib, etoricoxib, lumiracoxib, parecoxib and valdecoxib, as well as the pharmacologically acceptable derivatives (such as e.g. salts, esters, hydrates, polymorphs or stereoisomers) of these compounds.

Substances having good oral availability are preferred here.

According to aspect c of this invention “PDE5 inhibitor” refers to a selective PDE inhibitor, which inhibits preferentially the type 5 phosphodiesterase (PDE5) when compared to other known types of phosphodiesterase, e.g. type 1, 2, 3, 4 etc. (PDE1, PDE2, PDE3, PDE4, etc.). According to aspect c of this invention, a PDE inhibitor preferentially inhibiting PDE5 refers to a compound having a lower IC₅₀ for the type 5 phosphodiesterase compared to IC₅₀ for inhibition of other known type of phosphodiesterase (e.g. type 1, 2, 3, 4 etc), such as, for example, wherein the IC₅₀ for PDE5 inhibition is about factor 10 lower than the IC₅₀for inhibition of other known types of phosphodiesterase, e.g. type 1, 2, 3, 4 etc, and therefore is more potent to inhibit PDE5.

Methods to determine the activity and selectivity of a phosphodiesterase inhibitor are known to the person skilled in the art. In this connection it may be mentioned, for example, the methods described by Thompson et al. (Adv Cycl Nucl Res 10: 69-92, 1979), Giembycz et al. (Br J Pharmacol 118: 1945-1958, 1996) and the phosphodiesterase scintillation proximity assay of Amersham Pharmacia Biotech.

As PDE5 inhibitors within the meaning of aspect c of present invention may be mentioned, for example, without being limited to, those PDE5 inhibitors which are named expressis verbis as an example or described and/or claimed generically in the following patent applications and patents: WO 9626940, WO 9632379, EP 0985671, WO 9806722, WO 0012504, EP 0667345, EP 0579496, WO 9964004, WO 9605176, WO 9307124, WO 9900373, WO 9519978, WO 9419351, WO 9119717, EP 0463756, EP 0293063, WO 0012503, WO9838168, WO 9924433, DE 3142982 and U.S. Pat. No. 5,294,612.

Likewise, substances having good oral availability are preferred here.

Compounds which may be more worthy to be mentioned as examples of PDE5 inhibitors hereby are 3-ethyl-8-[2-(4-morpholinylmethyl)benzylamino]-2,3-dihydro-1H-imidazo[4,5-g]quinazoline-2-thione, 1-(2-chlorobenzyl)-3-isobutyryl-2-propylindole-6-carboxamide, 9-bromo-2-(3-hydroxypropoxy)-5-(3-pyridylmethyl)-4H-pyrido[3,2,1-jk]-carbazol-4-one, 4-(1,3-benzodioxol-5-ylmethylamino)-2-(1-imidazolyl)-6-methylthieno[2,3-d]pyrimidine, 6-(2-isopropyl-4,5,6,7-terahydropyrazolo[1,5-a]pyridin-3-yl)-5-methyl) -5-methyl-2,3,4,5-tetrahydropyridazin-3-one, 5-(4-methylbenzyl)-3-(1-methyl-4-phenylbutyl) -3,6-dihydro-[1,2,3]triazolo[4,5-d]pyrimidin-7-one, 3-(1-methyl-4-phenylbutyl)-5-pyridin-4-ylmethyl -3,6-dihydro-[1,2,3]triazolo[4,5-d]pyrimidin-7-one, 5-(4-bromobenzyl)-3-(1-methyl-4-phenylbutyl) -3,6-dihydro-[1,2,3]triazolo[4,5-d]pyrimidin-7-one, 5-benzyl-3-(1-methyl-4-phenylbutyl)-3,6-dihydro-[1,2,3]triazolo[4,5-d]pyrimidin-7-one, 5-(3,4-dimethoxybenzyl)-3-(1-methyl-4-phenylbutyl)-3,6-dihydro-[1,2,3]triazolo-[4,5-d]pyrimidin-7-one, 5-(3,4-dichlorobenzyl)-3-(1-methyl-4-phenylbutyl)-3,6-dihydro-[1,2,3]triazolo[4,5-d]pyrimidin-7-one, 5-biphenyl-4-ylmethyl-3-(1-methyl-4-phenylbutyl)-3,6-dihydro-[1,2,3]triazolo[4,5-d]pyrimidin-7-one, 5-(4-aminobenzyl)-3-(1-methyl-4-phenylbutyl)-3,6-dihydro-[1,2,3]triazolo[4,5-d]pyrimi-din-7-one, 5-(hydroxyphenylmethyl)-3-(1-methyl-4-phenylbutyl)-3,6-dihydro-[1,2,3]triazolo-[4,5-d]pyrimidin-7-one, 5-benzo[1,3]dioxol-5-ylmethyl-3-[1-methyl-4-phenylbutyl]-3,6-dihydro-[1,2,3]triazolo[4,5-d]pyrimidin-7-one, N-4-[3-(1-methyl-4-phenylbutyl)-7-oxo-6,7-dihydro -3H-[1,2,3]triazolo-[4,5-d]pyrimidin-5-ylmethyl]phenylacetamide, 5-benzoyl-3-(1-methyl-4-phenylbutyl)-3,6-dihydro-[1,2,3]triazolo[4,5-d]-pyrimidin-7-one, 3-(1-methyl-4-phenylbutyl)-5-[4-(morpholine -4-sulphinyl)benzyl]-3,6-dihydro[1,2,3]triazolo[4,5-d]pyrimidin-7-one, 3-(1-methyl-4-phenylbutyl)-5-[3-(morpholine-4-sulphonyl)benzyl]-3,6-dihydro[1,2,3]triazolo[4,5-d]pyrimidin-7-one, N-methyl-4-[3-(1-methyl-4-phenylbutyl)-7-oxo-6,7-dihydro-3H-[1,2,3]-triazolo-[4,5-d]pyrimidin-5-ylmethyl]-benzenesulphonamide, N-(2-dimethylaminoethyl)-4-[3-(1-methyl-4-phenylbutyl)-7-oxo-6,7-dihydro-3H-[1,2,3]triazolo[4,5-d]pyrimidin-5-ylmethyl]benzenesulphonamide, N-(2-hydroxyethyl)-4-[3-(1-methyl-4-phenylbutyl)-7-oxo-6,7-dihydro-3H-[1,2,3]triazolo[4,5-d]pyrimidin-5-ylmethyl]benzenesulphonamide, ethyl 1-[3-[3-(1-methyl-4-phenylbutyl)-7-oxo-6,7-dihydro-3H-[1,2,3]-triazolo-[4,5-d]pyrimidin-5-ylmethyl]benzenesulphonyl]piperidinecarboxylate, 3-(1-methyl-4-phenylbutyl)-5-[4-(4-methylpiperazin-1-sulphonyl)benzyl]-3,6-dihydro-[1,2,3]triazolo[4,5-d]pyrimidin-7-one, 5-benzo[1,3]dioxol-5-ylmethyl-3-[1-ethy-heptyl]-3,6-dihydro-[1,2,3]-triazolo[4,5-d]pyrimidin-7-one, 3-[1-(1-hydroxyethyl)-4-phenylbutyl]-5-[4-(morpholine-4-sulphonyl)benzyl]-3,6-dihydro-[1,2,3]triazolo[4,5-d]pyrimidin-7-one, 5-[6-fluoro-1-(phenylmethyl)-1H-indazol-3-yl]-2-furanmethanol, 1-benzyl-6-fluoro-3-[5-(hydroxymethyl)furan-2-yl]-1H-indazole, 2-(1H-imidazol-1-yl)-6-methoxy-4-(2-methoxyethylamino)quinazoline, 1-[[3-(7,8-dihydro-8-oxo-1H-imidazo[4,5-g]quinazolin-6-yl)-4-propoxyphenyl]sulphonyl]-4-methylpiperazine, 4-(3-chloro-4-methoxybenzylamino)-1-(4-hydroxypiperidin-1-yl)phthalazine-6-carbonitrile, 1-[6-chloro-4-(3,4-methylendioxybenzylamino)quinazolin-2-yl]piperidin-4-carboxylic acid, (6R, 12aR)-6-(1,3-benzodioxol-5-yl)-2-methyl-1,2,3,4,6,7,12,12a-octa-hydropyrazino[2′, 1′:6,1]pyrido[3,4-b]indole-1,4-dione (tadalafil), (6R, 12aR)-2,3,6,7,12,12a-hexahydro-2-methyl-6-(3,4-methylenedioxyphenyl)-pyrazino-[2′,1′:6,1]pyrido[3,4-b]indole-1,4-dione, 4-ethoxy-2-phenylcycloheptylimidazole, (6-bromo-3-methoxymethylimidazo[1,2-a]pyrazin-8-yl)methylamine, 8-[(phenylmethyl)thio]-4-(1-morpholinyl)-2-(1-piperazinyl)pyrimidino[4,5-d]pyrimidine, (+)-cis-5-methyl-2-[4-(trifluoromethyl)benzyl]-3,4,5,6a,7,8,9-octahydrocyclopent[4,5]imidazo[2,1-b]purin-4-one, cis-2-hexyl-5-methyl-3,4,5,6a,7,8,9,9a-octahydrocyclopent[4,5]imidazo[2,1-b]purin-4-one, 5-[2-ethoxy-5-(4-methyl-1-piperazinyl-sulphonyl)phenyl]-1-methyl-3-n-propyl-1,6-dihydro-7H-pyrazolo[4,3-d]pyrimidin-7-one (sildenafil), 1-[[3-(6,7-dihydro-1-methyl-7-oxo-3-propyl-1H-pyrazolo[4,3-d]pyrimidin-5-yl)-4-ethoxyphenyl]sulfonyl]-4-methylpiperazine, 2-(2-propoxyphenyl)purin-6(1H)-one, 2-(2-propoxyphenyl)-1,7-dihydro-5H-purin-6-one, methyl 2-(2-methylpyridin-4-ylmethyl)-1-oxo-8-(2-pyrimidinylmethoxy)-4-(3,4,5-trimethoxyphenyl)-1,2-dihydro-[2,7]naphthyridin-3-carboxylate, methyl 2-(4-aminophenyl)-1-oxo-7-(2-pyridinylmethoxy)-4-(3,4,5-trimethoxyphenyl)-1,2-dihydroisoquinoline-3-carboxylate, 2-[2-ethoxy-5-(4-ethylpiperazin-1-ylsulfonyl)phenyl]-5-methyl-7-propylimidazo[5,1-f][1,2,4]triazin-4(3H)-one (vardenafil), 3,4-dihydro-6-[4-(3,4-dimethoxybenzoyl)-1-piperazinyl]-2(1H)-quinolinone (vesnarinone), 1-cyclopentyl-3-methyl-6-(4-pyridyl)pyrazolo[3,4-d]pyrimidin-4(5H)-one, 1-cyclopentyl-6-(3-ethoxy-4-pyridinyl)-3-ethyl-1,7-dihydro-4H-pyrazolo[3,4-d]-pyrimidin-4-one, 6-o-propoxyphenyl-8-azapurin-6-one, 3,6-dihydro-5-(o-propoxyphenyl)-7H-v-triazolo[4,5-d]pyrimidin-7-one and 4-methyl-5-(4-pyridinyl)thiazole-2-carboxamide and the pharmaceutically acceptable derivatives of these compounds.

In the context of the present invention, unless otherwise stated, a pharmaceutically acceptable derivative of an active compound means a pharmaceutically acceptable salt or solvate (e. g. hydrate), a pharmaceutically acceptable solvate of such salt, a pharmaceutically acceptable N-oxide or a pharmaceutically acceptable salt or solvate of the latter.

PDE5 inhibitors, which are preferred in connection with aspect c of the invention are compounds selected from the group tadalafil, sildenafil, vardenafil, UK357903, E4010, E8010 and TA-1790, DA-8159 and solvates, polymorphs and/or the pharmacologically acceptable salts of these compounds.

A preferred PDE5 inhibitor is hereby sildenafil (which is 5-[2-ethoxy-5-(4-methylpiperazin-1-ylsulfonyl)phenyl]-1,6-dihydro-1-methyl-3-propylpyrazolo[4,3-d]pyrimidin-7-one), a pharmaceutically acceptable salt of sildenafil or a solvate of the pharmaceutically acceptable salt of sildenafil. In a preferred embodiment the PDE5 inhibitor is sildenafil citrate {the compound [1-[[3-(6,7-dihydro-1-methyl-7-oxo-3-propyl-1H-pyrazolo[4,3-d]pyrimidin-5-yl)-4-ethoxyphenyl]sulfonyl]-4-methylpiperazine citrate. The preparation of sildenafil is disclosed in EP 0463756.

Another preferred PDE5 inhibitor is hereby vardenafil [which is 1-{[3-(3,4-Dihydro-5-methyl-4-oxo-7-propylimidazo[5,1-f]-as-triazin-2-yl)-4-ethoxyphenyl]sulfonyl}-4-ethylpiperazine], a pharmaceutically acceptable salt of vardenafil or a solvate of the pharmaceutically acceptable salt of vardenafil. Examples of pharmaceutically acceptable salts of vardenafil are vardenafil hydrochloride, the trihydrate of vardenafil hydrochloride and vardenafil dihydrochloride. Vardenafil is known from WO99/24433.

Another preferred PDE5 inhibitor is hereby tadalafil [which is (6R,12aR)-2,3,6,7,12,12a-Hexahydro-2-methyl-6-[3,4-(methylenedioxy)phenyl]pyrazino[1′,2′:1,6]pyrido[3,4-b]indole-1,4-dione], a pharmaceutically acceptable salt of tadalafil or a solvate of the pharmaceutically acceptable salt of tadalafil. Tadalafil is known from WO95/19978.

The term “combination” according to this aspect c of this invention may be present as a fixed combination, a non-fixed combination or a kit-of-parts.

A “fixed combination” is defined as a combination wherein the said first active ingredient and the said second active ingredient are present together in one unit dosage or in a single entity. One example of a “fixed combination” is a pharmaceutical composition wherein the said first active ingredient and the said second active ingredient are present in admixture for simultaneous administration, such as in a formulation. Another example of a “fixed combination” is a pharmaceutical combination wherein the said first active ingredient and the said second active ingredient are present in one unit without being in admixture.

A “kit-of-parts” is defined as a combination wherein the said first active ingredient and the said second active ingredient are present in more than one unit. One example of a “kit-of-parts” is a combination wherein the said first active ingredient and the said second active ingredient are present separately. The components of the kit-of-parts may be administered separately, sequentially, simultaneously or chronologically staggered.

The present invention further relates to a pharmaceutical composition comprising

a first active ingredient, which is at least one COX-2 selective NSAID, such as e.g. a coxib, and
a second active ingredient, which is at least one PDE5 inhibitor, such as e.g. one or more of those mentioned herein,
and, optionally,
a pharmaceutically acceptable carrier or diluent,
for separate, sequential, simultaneous or chronologically staggered use in therapy.

The present invention further relates to a combination product comprising

a.) at least one COX-2 selective NSAID, such as e.g. a coxib, formulated with a pharmaceutically acceptable carrier or diluent, and
b.) at least one PDE5 inhibitor, such as e.g. one or more of those mentioned herein, formulated with a pharmaceutically acceptable carrier or diluent.

The present invention further relates to a kit-of-parts comprising

a preparation of a first active ingredient, which is a COX-2 selective NSAID, such as e.g. a coxib, and
a pharmaceutically acceptable carrier or diluent;
a preparation of a second active ingredient, which is a PDE5 inhibitor, and a pharmaceutically acceptable carrier or diluent;
for simultaneous, sequential, separate or chronologically staggered use in therapy.

Optionally, said kit comprises instructions for its use in therapy, e.g. to treat diseases responsive to COX-2 inhibition, such as inflammatory diseases.

Optionally, said kit comprises instructions for its use in therapy, e.g. to treat diseases responsive to COX-2 inhibition, such as inflammatory diseases, e.g. those from chronic nature, while reducing the risk of cardiovascular and/or renal side-effects associated with therapeutic use of selective COX-2 inhibitors alone.

The first and second active ingredient of the kit-of-parts according to this invention may be provided as separate formulations (i.e. independently of one another), which are subsequently brought together for simultaneous, sequential, separate or chronologically staggered use in combination therapy; or packaged and presented together as separate components of a combination pack for simultaneous, sequential, separate or chronologically staggered use in combination therapy.

The type of pharmaceutical formulation of the first and second active ingredient of the kit-of-parts according to this invention can be similar, i.e. both ingredients are formulated in separate tablets or capsules, or one active ingredient is formulated as tablet or capsule and the other is formulated for e.g. intravenous administration.

The present invention further relates to a combined preparation comprising at least one COX-2 selective NSAID and at least one PDE5 inhibitor for simultaneous, sequential or separate administration.

In this connection, the present invention further relates to combinations, compositions, formulations, preparations or kits according to the present invention having anti-inflammatory and cardio-protective properties.

In addition, the present invention further relates to the use of the combinations, compositions, formulations, preparations or kits according to aspect c of this invention in the manufacture of pharmaceutical products, such as e.g. commercial packages or medicaments, for the prevention or protection from cardiovascular and/or renal diseases associated with the use of COX-2 selective NSAIDs alone.

Further, the present invention further relates to the use of the combinations, compositions, formulations, preparations or kits according to aspect c of this invention in the manufacture of a pharmaceutical product, such as e.g. a commercial package or a medicament, for treating, preventing, or ameliorating diseases responsive to COX-2 inhibition, particularly those mentioned herein, such as e.g. inflammatory diseases, particularly those from chronic nature.

Further, the present invention further relates to the use of a PDE5 inhibitor in the manufacture of a pharmaceutical product for preventing cardiovascular diseases associated with the use of a COX-2 selective NSAID.

Further, the present invention relates to a method for inhibiting selectively (preferentially) COX-2 while reducing the risk of cardiovascular diseases comprising administering to a mammal in need thereof an effective amount of a COX-2 selective NSAID, such as e.g. a coxib, in combination with an effective amount of a PDE5 inhibitor.

Further, the present invention relates to a method for treating, preventing or ameliorating a disease responsive to COX-2 inhibition comprising administering to a mammal, including a human, in need thereof a therapeutically effective amount of a COX-2 selective NSAID, such as e.g. a coxib, in combination with a therapeutically effective amount of a PDE5 inhibitor.

Further, the present invention relates to a method for treating, preventing or ameliorating a disease responsive to COX-2 inhibition while reducing the risk of cardiovascular diseases comprising administering to a mammal, including a human, in need thereof a therapeutically effective amount of a COX-2 selective NSAID, such as e.g. a coxib, in combination with a therapeutically effective amount of a PDE5 inhibitor.

Further, the present invention relates to a method for treating, preventing or ameliorating a disease responsive to COX-2 inhibition while reducing the risk of cardiovascular and/or renal side effects associated with therapeutic use of COX-2 selective NSAIDs in a mammal in need thereof comprising administering to said mammal a therapeutically effective and tolerable amount of a compound from the class of COX-2 selective NSAIDs, such as e.g. from the coxib class, in combination with a therapeutically effective and tolerable amount of a PDE5 inhibitor.

Further, the present invention relates to a method for treating, preventing or ameliorating a disease responsive to COX-2 inhibition and reducing the risk of cardiovascular diseases in a mammal, including a human, in need of such treatment, prevention or amelioration and at risk of cardiovascular diseases comprising administering to said mammal a therapeutically effective and tolerable amount of a COX-2 selective NSAID, such as e.g. a coxib, in combination with a therapeutically effective and tolerable amount of a PDE5 inhibitor.

Further, the present invention relates to a method for preventing cardiovascular diseases, such as, for example, cardiovascular diseases associated a COX-2 selective NSAID, such as e.g. a coxib, in a mammal, including human, in need of such prevention comprising administering a therapeutically effective and tolerable amount of a PDE5 inhibitor in combination with said COX-2 selective NSAID to said mammal.

Further, the present invention further relates to a method for treating in combination therapy diseases responsive to COX-2 inhibition, such as e.g. those diseases or conditions mentioned herein, comprising administering a combination, composition, formulation, preparation or kit as described in aspect c herein to a patient in need thereof.

The present invention further relates to a commercial package comprising one or more COX-2 selective NSAIDs together with instructions for simultaneous, sequential or separate use with one or more PDE5 inhibitors in therapy, such as e.g. to treat a disease responsive to COX-2 inhibition while preventing cadio-renal side effects.

The present invention further relates to a commercial package comprising one or more PDE5 inhibitors together with instructions for simultaneous, sequential or separate use with one or more COX-2 selective NSAIDs in therapy, such as e.g. to treat a disease responsive to COX-2 inhibition while preventing cadio-renal side effects.

The compositions, combinations, preparations, formulations, kits or packages mentioned in the context of the combination therapy according to this invention may also include more than one of the COX-2 selective NSAIDs and/or more than one of the PDE5 inhibitors.

According to aspect c of this invention, suitable pharmaceutically acceptable salts refer to water-soluble and water-insoluble acid addition salts with acids such as, for example, hydrochloric acid, hydrobromic acid, phosphoric acid, nitric acid, sulfuric acid, acetic acid, citric acid, D-gluconic acid, benzoic acid, 2-(4-hydroxybenzoyl)-benzoic acid, butyric acid, sulfosalicylic acid, maleic acid, lauric acid, malic acid, fumaric acid, succinic acid, oxalic acid, tartaric acid, embonic acid, stearic acid, toluenesulfonic acid, methanesulfonic acid or 1-hydroxy-2-naphthoic acid, the acids being employed in salt preparation—depending on whether it is a mono- or polybasic acid and depending on which salt is desired—in an equimolar quantitative ratio or one differing there from. Furthermore, the active compounds mentioned can also be present as pure enantiomers or as enantiomer mixtures in any mixing ratio.

In addition, suitable pharmaceutically acceptable salts according to aspect c also refer to salts with bases, e.g. alkali metal (lithium, sodium, potassium) or calcium, aluminium, magnesium, titanium, ammonium, meglumine or guanidinium salts, which also employ bases in salt preparations in an equimolar quantitative ratio or deviations of it.

As a medicament (also referred to as pharmaceutical preparation hereinafter), the active agents mentioned in aspect c of this invention are either employed as such, or preferably in combination with suitable pharmaceutical auxiliaries and/or excipients, e.g. in the form of tablets, coated tablets, capsules, caplets, suppositories, patches (e.g. as TTS), emulsions, suspensions, gels, solutions or ointments. The pharmaceutical preparations typically comprises a total amount of active compound in the range from 0.05 to 99% w (percent by weight), more preferably in the range from 0.10 to 70% w, even more preferably in the range from 0.10 to 50% w, all percentages by weight being based on total preparation. By the appropriate choice of the auxiliaries and/or excipients, a pharmaceutical administration form (e.g. a delayed release form or an enteric form) exactly suited to the active compound and/or to the desired onset of action can be achieved.

The person skilled in the art is familiar with auxiliaries, adjuvants, diluents, carriers or excipients, which are suitable for the desired pharmaceutical preparations on account of his/her expert knowledge. In addition to solvents, gel-forming agents, tablet excipients and other active compound carriers, it is possible to use, for example, antioxidants, dispersants, emulsifiers, antifoams, flavor corrigents, preservatives, solubilizers, colorants or permeation promoters and complexing agents (e.g. cyclodextrins).

The active agents mentioned in aspect c may be administered in any acceptable mode of administration. A preferred method of administration of the active agents mentioned in aspect c is oral administration.

The amounts of the first and second active ingredients of the combinations, compositions or kits according to this invention may together comprise a therapeutically effective and tolerable amount for the treatment, prophylaxis or amelioration of a disease responsive to COX-2 inhibition, particularly one of those diseases mentioned herein, such as e.g. inflammatory diseases.

The dosage of the COX-2 selective NSAID administered is dependent on the species of warm-blooded animal (mammal), the body weight, age and individual condition, and on the form of administration. A unit dosage for oral administration to a mammal of about 50 to 70 kg may contain between about 5 and 1500 mg, e.g. from 100-1000 mg, preferably 200-800 mg of the active ingredient. COX-2 selective NSAID formulations in single dose unit form contain preferably from about 1% to about 90%, and formulations not in single dose unit form contain preferably from about 0.1% to about 20%, of the active ingredient. Single dose unit forms such as capsules, tablets or dragees contain e. g. from about 1 mg to about 1500 mg of the active ingredient.

In one embodiment, when the COX-2 selective NSAID is rofecoxib, it is preferred that the amount used is within a range of from about 0.15 to about 1.0 mg/kg per day, and even more preferably from about 0.18 to about 0.4 mg/kg per day.

In still another embodiment, when the COX-2 selective NSAID is etoricoxib, it is preferred that the amount used is within a range of from about 0.5 to about 5 mg/kg per day, and even more preferably from about 0.8 to about 4 mg/kg per day.

Further, when the COX-2 selective NSAID having an intrinsic PDE5-inhibitory component is celecoxib, it is preferred that the amount used is within a range of from about 1 to about 20 mg/kg per day, even more preferably from about 1.4 to about 8.6 mg/kg per day, and yet more preferably from about 2 to about 3 mg/kg per day. For example, doses of celecoxib for the treatment of osteoarthritis may be 100 to 200 mg per day, for the treatment of arthritis may be 200 to 400 mg per day, for the prevention of spontaneous adenomatopus polyps may be 400 mg a day, for adenoma prevention may be 400 to 800 mg a day, and for prevention of Alzheimer's disease may be 400 mg per day.

When the COX-2 selective NSAID is valdecoxib, it is preferred that the amount used is within a range of from about 0.1 to about 5 mg/kg per day, and even more preferably from about 0.8 to about 4 mg/kg per day.

In a further embodiment, when the COX-2 selective NSAID is parecoxib, it is preferred that the amount used is within a range of from about 0.1 to about 5 mg/kg per day, and even more preferably from about 1to about 3 mg/kg per day.

In a further embodiment, when the COX-2 selective NSAID is lumiracoxib, it is preferred that the amount used is within a range of from about 0.07 to about 10 mg/kg per day, and even more preferably from about 0.1 to about 7 mg/kg per day.

Tablet formulations for celecoxib, rofecoxib, lumiracoxib and valdecoxib are known under the tradenames Celebrex®, Vioxx®, Prexige® and Bextra® respectively.

The amount of PDE5 inhibitor, or a pharmaceutical acceptable derivative thereof which is required to achieve a therapeutic effect will, of course, vary with the particular compound, the route of administration, the subject under treatment, and the particular disorder or disease being treated. As a monotherapy, a PDE5 inhibitor is generally administered to adult humans by oral administration at a dose of 1 to 100 mg daily. As a monotherapy, sildenafil, vardenafil and tadalafil are generally administered to adult humans by oral administration at a dose of 1-100 mg daily.

Commercially available tablet formulations for sildenafil contain 25, 50 or 100 mg of sildenafil. Commercially available tablet formulations for vardenafil contain 5, 10 or 20 mg of vardenafil. Commercially available tablet formulations for tadalafil contain 10 or 20 mg of tadalafil.

Tablet formulations for sildenafil, tadalafil and vardenafil are commercially available under the tradenames Viagra®, Cialis® and Levitra® respectively.

Having described the invention in detail, the scope of the present invention is not limited only to those described characteristics or embodiments. As will be apparent to persons skilled in the art, modifications, analogies, variations, derivations, homologisations and adaptations to the described invention can be made on the base of art-known knowledge and/or, particularly, on the base of the disclosure (e.g. the explicite, implicite or inherent disclosure) of the present invention without departing from the spirit and scope of this invention as defined by the scope of the appended claims.

The following examples serve to illustrate the invention further without restricting it.

### EXAMPLES

### Biological Investigations

The phosphodiesterase-5 isoenzyme has been shown to be expressed in various cell types (e.g. smooth muscle cells and platelets) and tissues (Lin et al. Expression of three isoforms of cGMP-binding cGMP-specific phosphodiesterase (PDE5 ) in human penile cavernosum, Biochem Biophys Res Commun. (2000), 268: 628-635; Kotera et al. Genomic origin and transcriptional regulation of two variants of cGMP-binding cGMP-specific phosphodiesterases, Eur J Biochem (1999), 262: 866-873). In human up to now four splicing-variants of the pde5A-gene have been identified (PDE5A1, 5A2, 5A3, 5A4) which vary in tissue and cellular distribution. The substrate of phosphodiesterase-5 is cyclic-GMP which is hydrolyzed to GMP. Thus PDE5 controls the intracellular concentrations of cGMP, which is a major second messenger involved in the regulation of various cellular responses. For example cGMP has been shown to control proliferation and relaxation of smooth muscle cells and activity of platelets (aggregation, mediator release, adhesion) of various species (Osinski et al. Antimitogenic actions of organic nitrates are potentiated by sildenafil and mediated via activation of protein kinase, A. Mol Pharmacol. 2001, 59: 1044-1050, Wallis et al. Tissue distribution of phosphodiesterase families and the effects of sildenafil on tissue cyclic nucleotides, platelet function, and the contractile responses of trabeculae carneae and aortic rings in vitro, Am J Cardiol. (1999), 83: 3C-12C; Hirose et al., KF31327, a new potent and selective inhibitor of cyclic nucleotide phosphodiesterase 5, Eur J Pharmacol (2001) 431: 17-24). However the physiological effects of cGMP extents to numerous physiological effects, which is in part due to the activation protein kinase G by cGMP. Enhanced concentrations of cGMP are known to activate phosphodiesterase-2 and to inhibit phosphodiesterase-3. Since this two phosphodiesterases also hydrolyze cAMP the regulation of cGMP by PDE5-inhibitors also directly affects cAMP-signalling, thereby enhancing the complexity of effects induced by PDE5-inhibitors.

### Methods for Measuring Inhibition of PDE5 Activity

The PDE5A1 (GB no. AF043731) was cloned via PCR from Kidney cDNA. A 5′ fragment was amplified with OZ489 (5′-ATGGAGCGGGCCGGCCCCAGCTT -3′) and OZ 493 (5′-GTGTTCTGAATTCCCAAGCC-3′), a 3′ fragment was amplified with OZ 492 (5′-GGCTTGGGAATTCAGAACAC-3′) and OZ 490 (5′-TCAGTTCCGCTTGGCCTGGCCGCTT-3′). Both fragments were cloned into pCR2.1-Topo. Primers OZ 493 and OZ 492 have a nucleotide substitution that doesn't affect the translated sequence, but introduces an EcoRI cutting site. The 2 fragments were cut out of the cloning vector with EcoRI and subcloned together in a single ligation reaction into pBP9, resulting in the construct PZ 287. The ORF in PZ 287 is inverse to the promoter. For correct expression it was subcloned with Xmal/Sacl into pBacPak8.

The recombinant baculovirus was prepared by means of homologous recombination in SF9 insect cells. The expression plasmid was cotransfected with Bac-N-Blue (Invitrogen, Groningen, NL) or Baculo-Gold DNA (Pharmingen, Hamburg) using a standard protocol (Pharmingen, Hamburg). Wt virus-free recombinant virus supernatant was selected using plaque assay methods. After that, high-titre virus supernatant was prepared by amplifying 3 times. PDE was expressed in SF21 cells by infecting 2×10⁶ cells/ml with an MOI (multiplicity of infection) between 1 and 10 in serum-free SF900 medium (Life Technologies, Paisley, UK). The cells were cultured at 28° C. for 48-72 hours, after which they were pelleted for 5-10 min at 1000 g and 4° C.

The SF21 insect cells were resuspended, at a concentration of approx. 10⁷ cells/ml, in ice-cold (4° C.) homogenization buffer (20 mM Tris, pH 8.2, containing the following additions: 140 mM NaCl, 3.8 mM KCl, 1 mM EGTA, 1 mM MgCl₂, 10 mM β-mercaptoethanol, 2 mM benzamidine, 0.4 mM Pefablock, 10 μM leupeptin, 10 μM pepstatin A, 5 μM trypsin inhibitor) and disrupted by ultrasonication. The homogenate was then centrifuged for 10 min at 1000× g and the supernatant was stored at −80° C. until subsequent use (see below). The protein content was determined by the Bradford method (BioRad, Munich) using BSA as the standard.

PDE5A1 activity is inhibited by the said compounds in a modified SPA (scintillation proximity assay) test, supplied by Amersham Biosciences (see procedural instructions “phosphodiesterase [3H]cAMP SPA enzyme assay, code TRKQ 7090”), carried out in 96-well microtitre plates (MTP's). The test volume is 100 μl and contains 20 mM Tris buffer (pH 7.4), 0.1 mg of BSA (bovine serum albumin)/ml, 5 mM Mg²⁺, 0.5 μM cGMP (including about 50,000 cpm of [3H]cGMP as a tracer), 1 μl of the respective substance dilution in DMSO and sufficient recombinant PDE (1000× g supernatant, see above) to ensure that 10-20% of the cGMP is converted under the said experimental conditions. The final concentration of DMSO in the assay (1% v/v) does not substantially affect the activity of the PDE investigated. After a preincubation of 5 min at 37° C., the reaction is started by adding the substrate (cGMP) and the assay is incubated for a further 15 min; after that, it is stopped by adding SPA beads (50 μl). In accordance with the manufacturer's instructions, the SPA beads had previously been resuspended in water, but were then diluted 1:3 (v/v) in water; the diluted solution also contains 3 mM IBMX to ensure a complete PDE activity stop. After the beads have been sedimented (>30 min), the MTP's are analyzed in commercially available luminescence detection devices. The corresponding IC₅₀ values of the compounds for the inhibition of PDE activity are determined from the concentration-effect curves by means of non-linear regression.

Representative inhibitory values determined in the aforementioned assay follow from the following Table A.

TABLE A
Inhibition of the PDE5 activity

| Compound | μM |
|---|---|
| Diclofenac | >100 |
| Aspirin | >100 |
| Celecoxib | 16 |
| Rofecoxib | >100 |
| Lumiracoxib | >100 |
| Valdecoxib | >100 |
| Parecoxib | >100 |
| Etorecoxib | >100 |

### Method: Perfused Guinea-Pig Langendorff Heart

(Modification: Bolus Injection into the Perfusion Flow)

Guinea-pigs (male, Dunkin Hartley, 400-500 g; Charles River, Sulzfeld, Germany) kept in Macrolon® cages (type IV) with free access to food (maintenance diet no. 3418, Provimi-Kliba, CH-4303 Kaiseraugst) and tap water, were killed by cervical dislocation and the thoracic cavity was opened. The heart was rapidly excised and perfused with Krebs-Henseleit solution at a constant pressure of 80 cm H₂O (62 mmHg) via retrograde cannulation of the aorta in a Langendorff apparatus. The Krebs-Henseleit solution was composed as follows (mM: NaCl 118, KCl 4.7, CaCl₂ 1.9, MgSO₄ 1.2, KH₂PO₄ 1.2, NaHCO₃ 25.0, and glucose 5.0, at 37° C., gassed with 95% O₂/5% CO₂, v/v). Through an incision in the left atrium, a water-filled balloon catheter connected to a Statham P 23 Db pressure transducer was introduced into the left ventricle and preloaded to a pressure of 40 mmHg, mimicking the diastolic pressure.

Precoronary perfusate flow (CF, at an initial average passive flow of 11-13 ml/min) was measured using an electromagnetic flow meter. The change in left ventricular isovolumetric pressure amplitude (LVP) calculated by subtracting the diastolic from the systolic pressure, the rate of maximal left ventricular pressure rise (dP/dtₘₐₓ), and the rate of the spontaneously beating heart (HR) were continuously recorded and monitored by a data acquisition system (Notocord, hem version 3.3; F-78290 Croissy).

Following at least 1 h of perfusion and stabilization of all cardiac parameters, the test drug is injected as 100 μl bolus within 2 s directly into the perfusion tube connected to the aortic inflow and increased threefold after the previous dose has produced its coronary dilatory response and the perfusion flow has returned to pre-drug values again. Thee drug-induced change in cardiac parameters is expressed as percentage of the initial pre-drug values.

The following scheme shows the influence of PDE4, 5 and COX Inhibitors on coronary flow in GP Langendorff hearts:

1.-4. (canceled)

5. A method for preventing cardiovascular diseases in a mammal in need of such prevention comprising administering to said mammal a therapeutically effective and tolerable amount of a compound from the class of COX-2 selective NSAIDs, having an intrinsic PDE5-inhibitory component.

6. A method for treating, preventing or ameliorating a disease responsive to COX-2 inhibition in a subject by administering to a patient in need thereof a compound from the class of COX-2 selective NSAIDs, which method comprises administering to a mammal in need thereof a therapeutically effective and tolerable amount of a compound from the class of COX-2 selective NSAIDs, having an intrinsic PDE5-inhibitory component.

7. A method for treating, preventing or ameliorating a disease responsive to COX-2 inhibition while reducing the risk of cardiovascular diseases in a mammal in need thereof comprising administering to said mammal a therapeutically effective and tolerable amount of a compound from the class of COX-2 selective NSAIDs, having an intrinsic PDE5-inhibitory component.

8. A method for treating, preventing or ameliorating a disease responsive to COX-2 inhibition in a patient group in need of such treatment, prevention or amelioration and at risk of cardiovascular diseases comprising administering to said mammal a therapeutically effective and tolerable amount of a compound from the class of COX-2 selective NSAIDs, having an intrinsic PDE5-inhibitory component.

9. The method according to claim 6, wherein the compound from the class of COX-2 selective NSAIDs having an intrinsic PDE5-inhibitory component is celecoxib.

10. A combination comprising
a first active ingredient, which is at least one compound from the class of COX-2 selective NSAIDs, and
a second active ingredient, which is at least one PDE5 inhibitor,
for separate, sequential, simultaneous or chronologically staggered use in therapy.

11. (canceled)

12. A method for treating, preventing or ameliorating a disease responsive to COX-2 inhibition in a mammal in need thereof comprising administering to said mammal a therapeutically effective and tolerable amount of a compound from the class of COX-2 selective NSAIDs, in combination with a therapeutically effective and tolerable amount of a PDE5 inhibitor.

13. A method for treating, preventing or ameliorating a disease responsive to COX-2 inhibition while reducing the risk of cardiovascular and/or renal side effects associated with therapeutic use of COX-2 selective NSAIDs in a mammal in need thereof comprising administering to said mammal a therapeutically effective and tolerable amount of a compound from the class of COX-2 selective NSAIDs, in combination with a therapeutically effective and tolerable amount of a PDE5 inhibitor.

14. A method for preventing cardiovascular diseases associated with the use of a compound from the class of COX-2 selective NSAIDs, in a mammal in need of such prevention comprising administering a therapeutically effective and tolerable amount of a PDE5 inhibitor in combination with said compound from the class of COX-2 selective NSAIDs.

15. The combination according to claim 10 wherein the compound from the class of COX-2 selective NSAIDs is selected from the group consisting of celecoxib, cimicoxib, etoricoxib, firocoxib, lumiracoxib, parecoxib, rofecoxib, tilnacoxib and valdecoxib.

16. (canceled)

17. The combination according to claim 10 wherein the PDE5 inhibitor is selected from sildenafil, tadalafil and vardenafil.

18. A method for inhibiting selectively or preferentially COX-2 while reducing the risk of cardiovascular diseases in a mammal comprising administering to said mammal
either an effective amount of a compound from the class of COX-2 selective NSAIDs, which has an intrinsic PDE5-inhibitory component;
or an effective amount of a compound from the class of COX-2 selective NSAIDs, in combination with an effective amount of a PDE5 inhibitor.

19. A method for identifying a compound from the class of COX-2 selective NSAIDs useful for treating a disease responsive to COX-2 inhibition while reducing the risk of cardiovascular diseases, which method comprises determining for said compound from the class of COX-2 selective NSAIDs the existence of an intrinsic PDE5-inhibitory component.

20. A method of treating a disease responsive to COX-2 inhibition in a patient comprising administering to said patient a therapeutically effective amount of a compound from the class of COX-2 selective NSAIDs, selected by determining PDE5 inhibitory activity of said compound from the class of COX-2 selective NSAIDs, wherein the compound from the class of COX-2 selective NSAIDs that is selected for administration to said patient inhibits PDE5 activity less than about 50 μM.

21. The method according to claim 6, wherein the disease responsive to COX-2 inhibition is selected from the group consisting of
chronic inflammatory diseases degenerative dementia; and certain cancerous or pre-cancerous diseases.

22. The method according to claim 6, wherein the cardiovascular diseases are cardiovascular or thromboembolic adverse events or cerebrovascular accidents.
